(19) United States
(12) Patent Application Publication
(10) Pub. No.: US 2019/0105418 A1
(43) Pub. Date: Apr. 11, 2019

(54) METHOD AND DEVICE FOR SANITIZING LOW MOISTURE ENVIRONMENTS

(71) Applicant: PURE Bioscience, Inc., El Cajon, CA (US)

(72) Inventors: Dan JAQUES, Grand Rapids, MI (US); Clint BALL, Collinsvile, IL (US); Thomas A. MYERS, Redwood City, CA (US)

(21) Appl. No.: 16/153,001

(22) Filed: Oct. 5, 2018

Related U.S. Application Data
(60) Provisional application No. 62/569,145, filed on Oct. 6, 2017.

(51) Int. Cl. A61L 2/22; B08B 3/02; B08B 3/08; B08B 3/10; B08B 9/08

(52) U.S. Cl. CPC A61L 2/22; B08B 3/02; B08B 2209/08; B08B 3/102; B08B 9/08; B08B 3/08

(57) ABSTRACT

Certain aspects of the present disclosure relate to methods and apparatus for providing microbial sanitation. For example, a system for providing microbial sanitation generally includes a sanitizer configured to sanitize at least one of an environment or a target, a carrier configured to mix with the sanitizer creating a liquid mixture of sanitizer and carrier, and a sanitizer delivery device configured to hold the liquid mixture, apply pressurized air to the liquid mixture, and release the pressurized liquid mixture through a nozzle that creates a mist with micron-sized liquid particles that contact one or more of the environment or the target for a predetermined misting time based on at least one of the environment or the target.

### CLAIM OF PRIORITY UNDER 35 U.S.C. § 119

The present Application for Patent claims benefit of U.S. Provisional Patent Application Ser. No. 62/569,145, filed Oct. 6, 2017, assigned to the assignee hereof and hereby expressly incorporated by reference herein.

### BACKGROUND

### Field of the Disclosure

The present disclosure relates generally to sanitizing systems, and more particularly, to methods and apparatus for implementing sanitizing systems where access and moisture are restricted.

### Description of Related Art

There are many locales where systems and methods may be implemented in an attempt to help control microorganisms. For example, one or more locations where such implementations are provided include locations utilized by the food and medical industries. These areas needing sanitation can have particular limitations which restrict the sanitation process including reentry requirements, residual toxicity, and/or allowable moisture. Some of these restrictions may relate to regulatory requirements.

A number of approaches are provided in the literature for implementing sanitation related systems and methods that may help control microorganisms. Providing sanitation has been the focus of much industrial development and has produced numerous chemicals and devices that target microbial control. Currently, these technologies are under pressure to improve their ability to provide sanitation properties and microbial control. This pressure stems from, at least in part, customers that are increasingly less tolerant of sanitation issues in any environment. However, many of these approaches are currently tailored to unrestricted environments but are not tailored to environments that are more restrictive. Accordingly, novel and effective approaches are needed to meet the sanitation requirements of these special environments.

Thus, as the demand for sanitation technologies continues to increase, there exists a desire for further improvements in sanitation technology that may provide microbial control. These improvements may be applicable to other technologies and the industries that employ these technologies.

### BRIEF SUMMARY

The systems, methods, and devices of the disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include improved sanitation including, for example, microorganism control.

Certain aspects provide a system for providing microbial sanitation. The system generally includes a sanitizer configured to sanitize at least one of an environment or a target, a carrier configured to mix with the sanitizer creating a liquid mixture of sanitizer and carrier, and a sanitizer delivery device configured to hold the liquid mixture, apply pressurized air to the liquid mixture, and release the pressurized liquid mixture through a nozzle that creates a mist with micron-sized liquid particles that contact one or more of the environment or the target for a predetermined misting time.

Certain aspects provide a sanitizer delivery device. The sanitizer delivery device generally includes a sanitizer and carrier mixture reservoir configured to hold a sanitizer and carrier mixture in liquid form that is configured to be misted, a compressed air source that is configured to apply compressed air to the sanitizer and carrier mixture, and at least one nozzle that is configured to create a mist from the sanitizer and carrier mixture as the sanitizer and carrier mixture passes through the nozzle under pressure from the compressed air source that is applied.

Certain aspects provide a method for sanitizing an environment. The method generally includes providing a misting solution that includes a sanitizer and misting the misting solution into the environment such that a mist of the misting solution includes micron-sized particle mist for a predetermined misting time.

Aspects generally include methods, apparatus, systems, computer readable mediums, and processing systems, as substantially described herein with reference to, and as illustrated by, the accompanying drawings.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a block diagram conceptually illustrating an example system for sanitizing an environment, in accordance with certain aspects of the present disclosure.

FIG. 2 is a block diagram conceptually illustrating an example system for sanitizing a target, in accordance with certain aspects of the present disclosure.

FIGS. 3A and 3B are diagrams illustrating examples of an air pump that serves air to a sanitizer delivery device, in accordance with certain aspects of the present disclosure.

FIGS. 4A and 4B are diagrams illustrating examples of an air pump that serves multiple sanitizer delivery devices, in accordance with certain aspects of the present disclosure.

FIG. 5 is a block diagram conceptually illustrating an example system for sanitizing an environment that includes multiple features, in accordance with certain aspects of the present disclosure.

FIG. 6 is a block diagram conceptually illustrating an example system for sanitizing an environment that includes multiple features, in accordance with certain aspects of the present disclosure.

FIG. 7 is a block diagram conceptually illustrating an example system for sanitizing an environment and/or target that includes multiple features, in accordance with certain aspects of the present disclosure.

FIG. 8A illustrates a system for sanitizing an environment and/or target that includes multiple features, in accordance with certain aspects of the present disclosure.

FIG. 8B illustrates a system sanitizing an environment and/or target outputting a mist, in accordance with certain aspects of the present disclosure.

FIG. 9 illustrates example operations for sanitizing an environment, in accordance with aspects of the present disclosure.

FIG. 10 is a table that contains sanitation results, in accordance with aspects of the present disclosure.

FIG. 11 illustrates an environment within which the results in FIG. 10 were collected, in accordance with aspects of the present disclosure.

FIGS. 12A, 12B, 12C, 12D, and 12E show testing locations were a swab was used on targets in an environment, in accordance with aspects of the present disclosure.

FIGS. 13A, 13B, and 13C show testing environments that include different size truck containers, in accordance with aspects of the present disclosure.

FIG. 14 is a table showing testing results, in accordance with aspects of the present disclosure.

FIGS. 15A and 15B are tables that show other resting results, in accordance with aspects of the present disclosure.

FIG. 16 is a table that shows results from another test, in accordance with aspects of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Aspects of the present disclosure provide apparatus, methods, processing systems, and/or computer readable mediums for providing sanitation in one or more environments. For example, in accordance with one or more cases, a multiple element system may be provided that allows for the sanitation of several classes of restricted environments. This system may include a sanitizer delivery device, an environmentally compatible sanitizer, and an appropriate carrier to concentrate the sanitizer on surfaces to be sanitized. These elements can be used independently/singularly or in any combination to provide microbial control and sanitation.

In one or more cases, a system may be provided that can output a particle mist with micron-sized droplets into a desired environment. The particle mist may include a sanitizer that controls pathogens in the select environment. Examples of the sanitizer include silver ions and/or plasma activated water. One example of one such environment includes a freeze tunnel where excess water would require an extensive interruption is processing. In accordance with other embodiments, a similar fine mist may be used to sanitize truck trailers and/or shipping containers between uses without a need to rinse. Other environments such as storage facilities, processing environments, or other similar environments where restrictive access rights are present may also be sanitized using the above-described delivery system and/or sanitizer.

The following description provides examples and is not limiting of the scope, applicability, or examples set forth in the claims. Changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim. The word “exemplary” is used herein to mean “serving as an example, instance, or illustration.” Any aspect described herein as “exemplary” is not necessarily to be construed as preferred or advantageous over other aspects.

### Example of Sanitizers

In accordance with one or more cases, a number of different factors may be taken into account when selecting sanitizers. Some of the factors help determine the materials that are environmentally compatible sanitizers. For example, one or more examples of such factors may include, but is not limited to, selective toxicity, regulatory requirements, and/or chemical compatibility. There are several common sanitizers that are useful for certain situations but are not compatible with many environments. For example, ethylene oxide may be used to sterilize medical devices but may be too toxic for use in areas where people need to enter. Quaternary ammonia materials may be used to sanitize many food processing lines but require removal. Chlorine materials, including but not limited to, hypochlorites and chlorine dioxide, are quite corrosive. For example, chlorine gas needs to be purged from the environment before an area may be considered to be safe for people to access. Active oxygen compounds such as peroxides or ozone can also be considered. However, these are corrosive and present toxicity hazards as well. There are other compatibility issues in various situations.

Accordingly, when sanitation or sterilization is needed, those skilled in the art may do that which is necessary such as including procedures to rinse, remove, or flush out sanitizing materials as described above. And in other situations, sanitation procedures may be postponed until that which needs sanitizing is moved to a less restrictive environment where a known material can be used. Accordingly, other materials are desired that have the ability to sanitize without one or more of the side effects or drawbacks as described above with the current material options.

Thus, in accordance with one or more cases, a solution of silver ions may be used as a sanitizer. For example, silver ions solutions particularly those stabilized with citric or lactic acids may be used as a sanitizer. Such solutions have tremendous lethality and are effective at low concentrations depending on the application. PURE Hard Surface™ offered by PURE Bioscience, Inc. is an example. Environmental Protection Agency (EPA) approved concentration at 30 ppm. The utility of such solutions may be greatly enhanced by the very low human toxicity. In many applications including food contact surfaces, no rinsing is required. Further, such solutions are effective against a wide range of organisms including bacteria, yeast and molds, and many viruses. In some situations, there may be residual antimicrobial activity which can be desirable. These features give such silver ion solutions great compatibility. This compatibility is illustrated in the specific examples disclosed below.

In accordance with one or more cases, another material that may be used as a sanitizer is plasma activated water. Plasma activated water is another highly compatible sanitizer class. This sanitizer class includes solutions containing nitrogen and sulfur compounds which yield especially lethal species with short lives. These materials achieve compatibility with very short half-lives. The toxicity for microorganisms is high with relatively low toxicity to humans. The energy requirement at the point of application is a limitation in some situations where the electrical grid is inaccessible and one must generate power locally.

Additionally, other similar materials that present with similar properties to silver ion solutions and plasma activated water may also be used by one or more cases to provide sanitization in restricted environments. Particularly, a sanitizer that is highly lethal to microorganisms with low human toxicity, that is effective at relatively safe concentrations, and where residues are manageable may be compatible with many special environments.

### Example of Sanitizer Delivery Devices

In accordance with one or more cases, a sanitizer delivery device may be provided that generates a mist using at least one sanitizer within a select environment where sanitation is desired for application on one or more targets. The sanitizer may be, for example, one or more of those described herein. A carrier may also be included with the sanitizer for misting and target adherence. In one or more cases, the sanitizer delivery device may be a highly variable element depending on the sanitation target and/or environment.

For example, in some cases, the sanitizer delivery device may include a portable spray unit with a timer or a remote control. This sanitizer delivery device may be provided to generate and apply the select sanitizer to the select target within an environment. The sanitizer delivery device may be provided with one or more nozzles that are configured to deliver fine mists in enclosed areas where access is possible. In some cases, it may be more effective to use gravity to disperse the mist which can be accomplished by raising the nozzles within the space being sanitized. The one or more nozzles in this sanitizer delivery device may be selected to provide particles that have sizes ranging from 5 microns up to 25 microns at a normal operating pressure of an included pump. In accordance with other cases, alternative configurations are possible such as those that use compressed air as a source of energy for mist dispersal.

Alternatively, given the low toxicity and stability of the silver ions, a hand sprayer may be used to spray select targets in an environment. Further, alternative designs can be fabricated as backpack units or as more traditional compressed bottle sprayers. However, such systems may not provide optimal droplet size for all environments and targets. Further, in one or more cases, such systems may also result in the use of more material than may be needed or in some situations using more carrier than a particular environment may tolerate. Accordingly, the sanitizer delivery device may be designed to include a nozzle, pressure source, carrier, and sanitizer that when combined provide a mist with a certain particulate size and disbursement abilities that are tailored for the particular target and/or environment that is being treated.

In accordance with other cases, the sanitizer delivery device may be a fixed installation within an environment such as, but not limited to, a freezer tunnel, a trailer, a cargo container, an operations room that includes one or more pieces of industrial equipment (which may also be targets for sanitation), and/or other restrictive environments. An example of a fixed installation is a freezer tunnel where periodic sanitation is desirable, where product could be exposed to the sanitizer as an incidental additive, and where ice deposits would hinder operations may include specifically installed nozzles in the environment that are configured to disperse droplets of a specific size at a specific flow and speed for that environment. It may be critical to minimize carrier usage in such situations. Sanitation of the environment with any one or more of these systems may be completed with only a modest pause in production.

One skilled in the art can readily identify numerous other nozzle configurations and delivery mechanisms that will deliver the desired small particle size. However, there are two additional classes that merit consideration. It can be advantageous to use air-assisted nozzles to reduce solution usage and deliver the desired small particles. This air can be used to increase volume without increasing solution usage to allow more surface coverage, an example of which includes a sanitizer delivery device that may be provided for use with truck trailers as described herein. In accordance with other cases, when attempting to use plasma activated water as a sanitizer, operations may be included that nebulize the carrier prior to plasma activation which may require somewhat larger delivery locations as compared to simple nozzle placement.

### Example of Carriers

In accordance with one or more cases, a third element that may be provided for implementing the use of a sanitizer and sanitizer delivery device as discussed above is a carrier. For example, by using a carrier such as water, the sanitizer may be localized in droplets which will adhere to surfaces rather than remain on the whole volume of the sanitation target. In accordance with other cases, other liquids may be utilized as a carrier. The small particles allow for a very thin coating of the surface delivering the maximal sanitation where is it needed. This element distinguished the cases disclosed herein from the well-known fumigation techniques where chemical particulate is directly released into the air in a gaseous state.

### Example of Sanitizing Systems

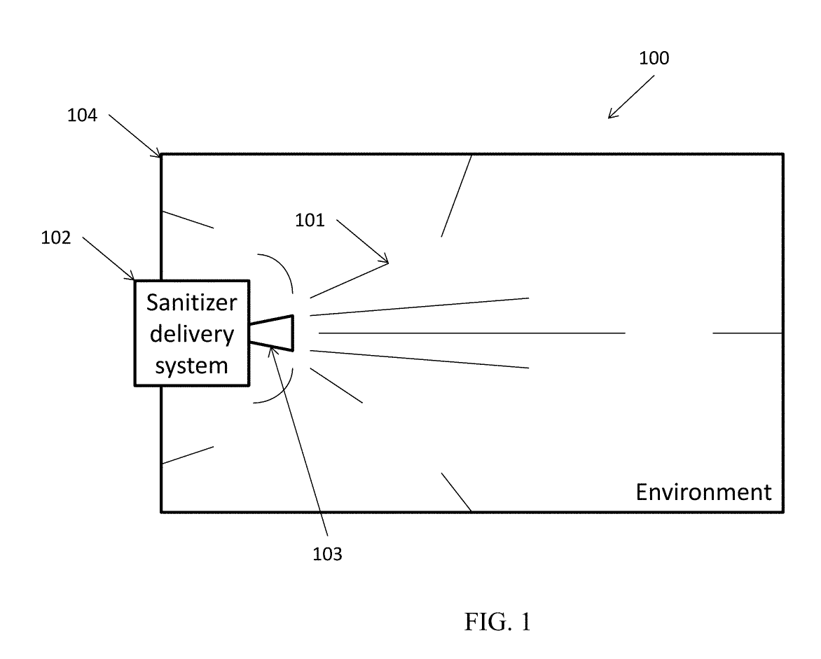

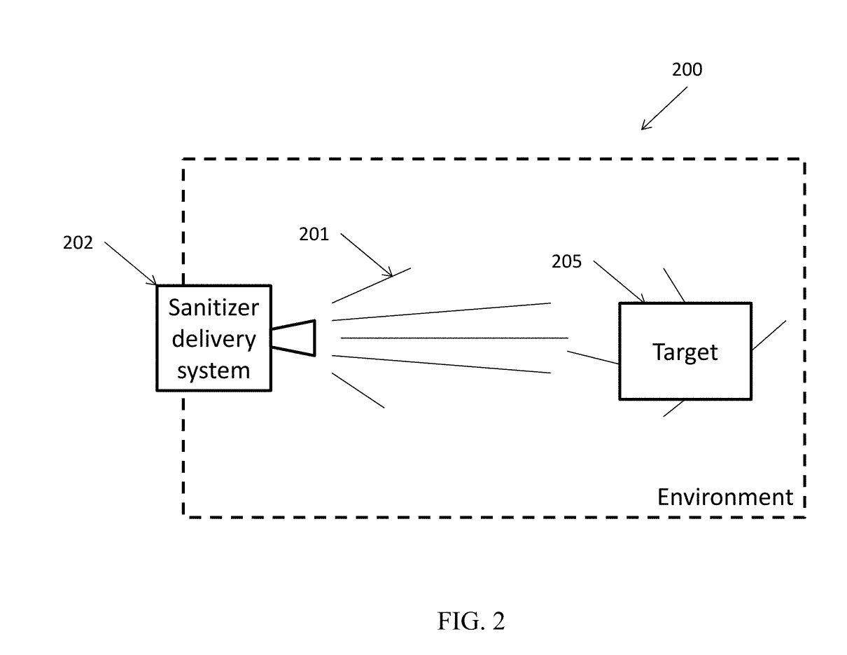

FIG. 1 is a block diagram conceptually illustrating an example system 100 for sanitizing an environment 104, in accordance with certain aspects of the present disclosure. As shown the system 100 may include a sanitizer delivery device 102. The sanitizer delivery device 102 is configured to release a mist 101 using a nozzle 103 into an environment 104 such that the mist 101 comes into contact with all surfaces of the environment 104 as shown. FIG. 2 is a block diagram conceptually illustrating an example system 200 for sanitizing a target 205, in accordance with certain aspects of the present disclosure. As shown, the system 200 includes a sanitizer delivery device that releases a mist 201 that comes into contact with many if not all surfaces of the target 205.

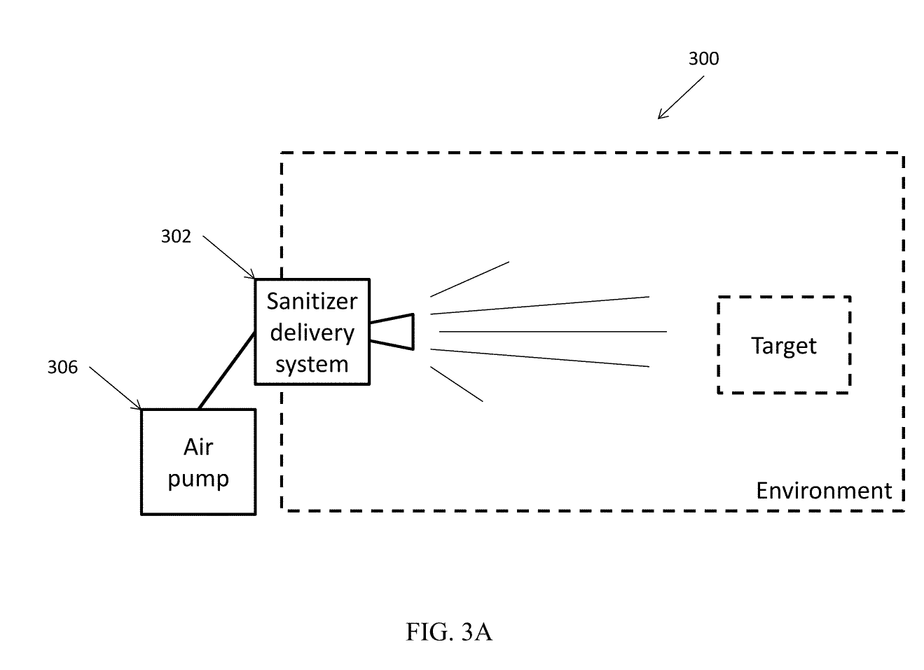

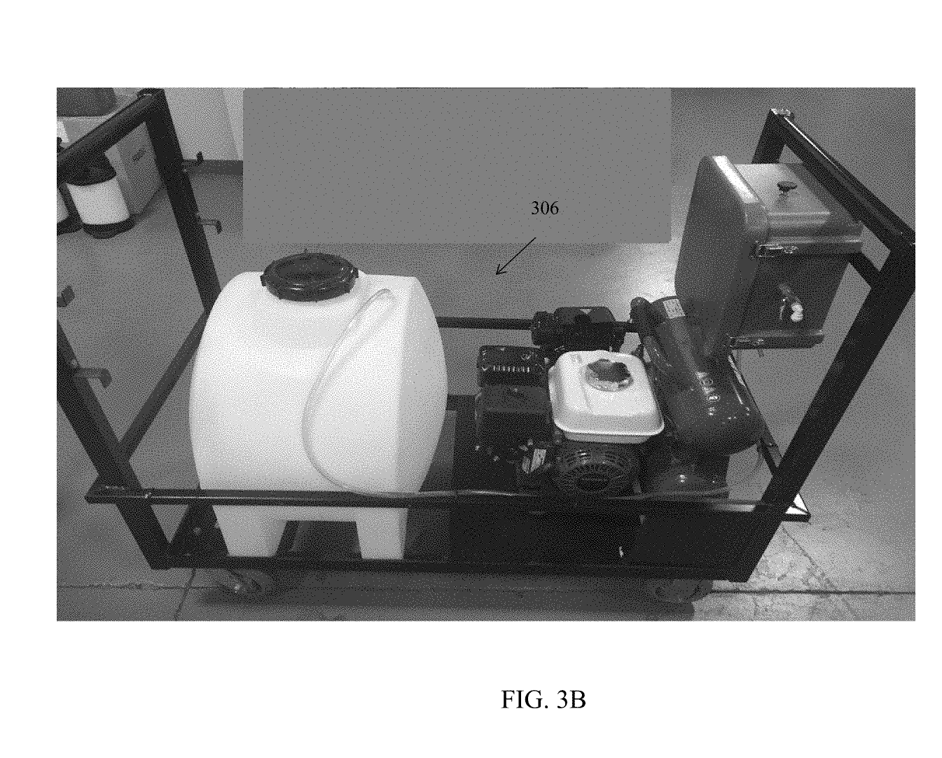

FIGS. 3A and 3B are diagrams illustrating examples of an air pump 306 that serves air to a sanitizer delivery device 302 to form an overall system 300, in accordance with certain aspects of the present disclosure. As shown, the air pump 306 may be provided separate from the sanitizer delivery device 302. A specific example of an air pump 306 is provided in FIG. 3B which shows a specific compatible air pump 306. A method of using the air pump 306 as shown in FIG. 3B may include, but is not limited to: 1. Ensure compressor has fuel; 2. Hook Compressor to Truck Misting Unit; 3. Turn on Compressor; 4. Place Misting unit OR Misting Stand inside of truck; 5. Select truck size; and 6. Hit start and cycle will run for proper application.

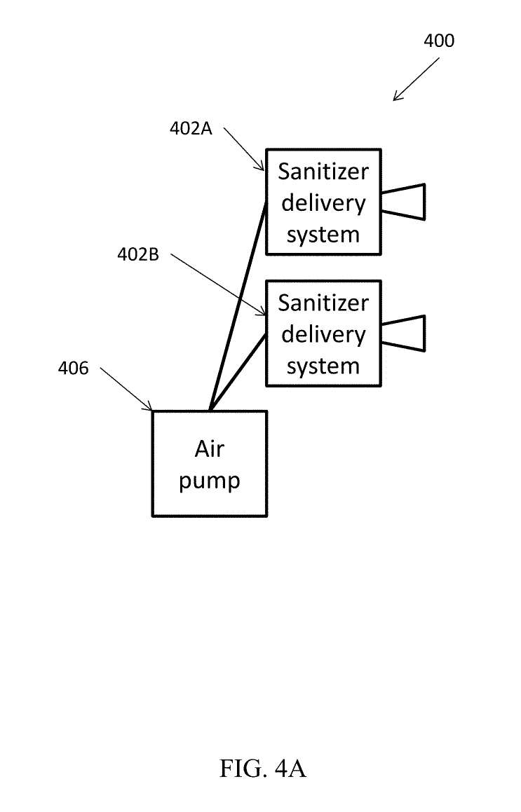

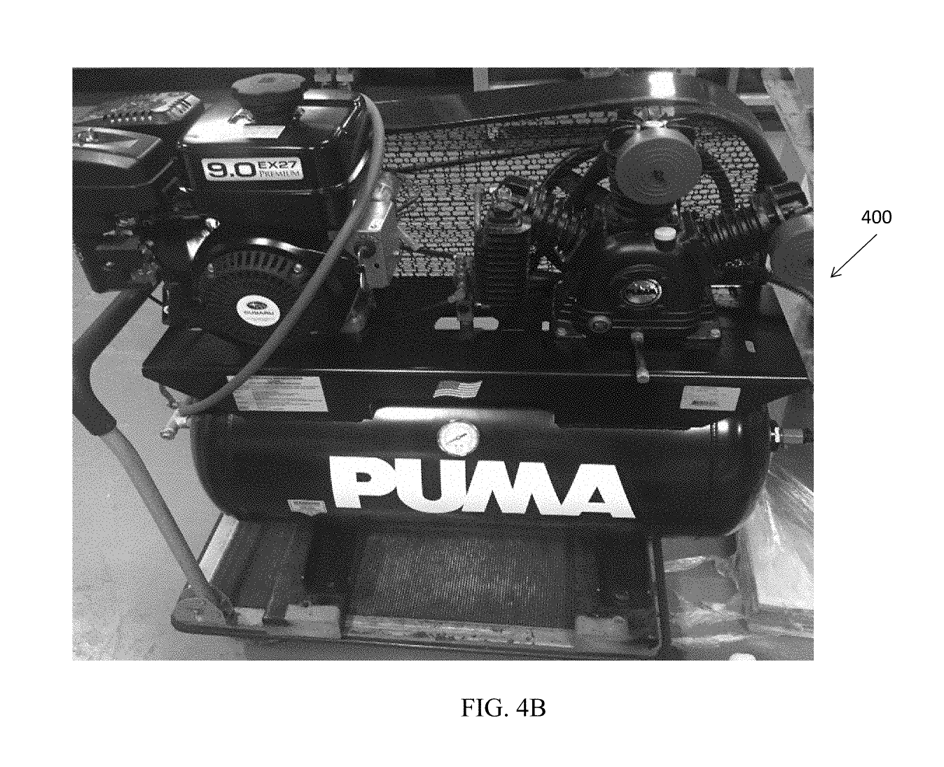

FIGS. 4A and 4B are diagrams illustrating examples of an air pump 406 that serves multiple sanitizer delivery devices 402A and 402B, in accordance with certain aspects of the present disclosure. FIG. 4B shows a specific example of an air pump 406. A method of using the air pump 406 as shown in FIG. 4B may include, but is not limited to: 1. Ensure compressor has fuel; 2. Hook Compressor to one or two Truck Misting Unit(s); 3. Turn on Compressor; 4. Place Misting unit(s) OR Misting Stand(s) inside of truck; 5. Select truck size; and 6. Hit start and cycle will run for proper application.

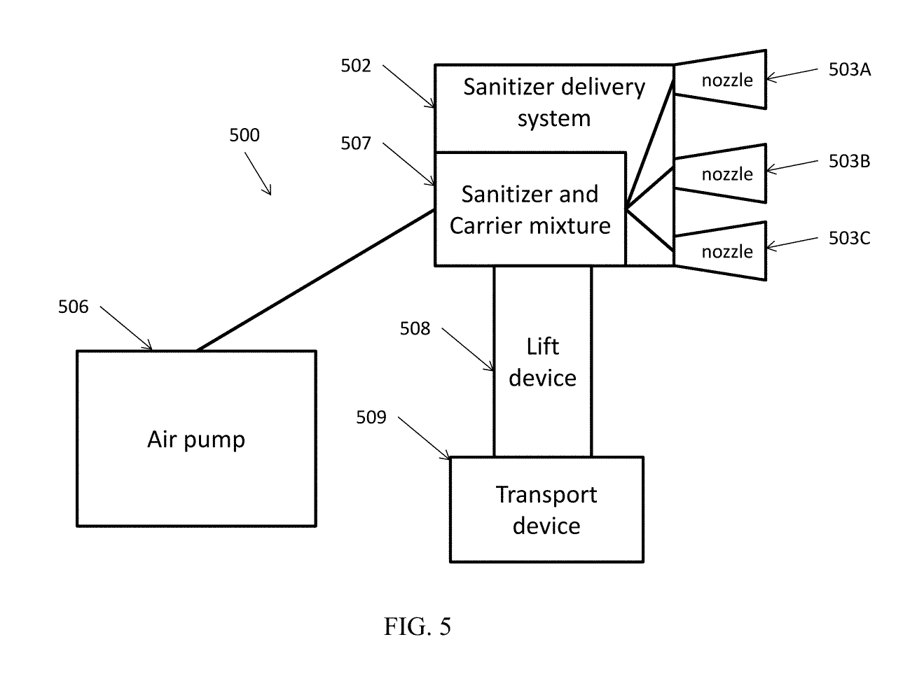

FIG. 5 is a block diagram conceptually illustrating an example system 500 for sanitizing an environment that includes multiple features, in accordance with certain aspects of the present disclosure. Specifically, the sanitizer delivery device 502 may include a sanitizer and carrier mixture 507 that may be in the form of a liquids tank. The sanitizer delivery device 502 may also include a plurality of nozzles 503A, 503B, and 503C that are configured to create mist particulate at the micron level. The system 500 may also include a lift device 508 for lifting the sanitizer delivery device 502 and a transport device 509 for helping move the device around. Finally, the system 500 includes an air pump 506 as well.

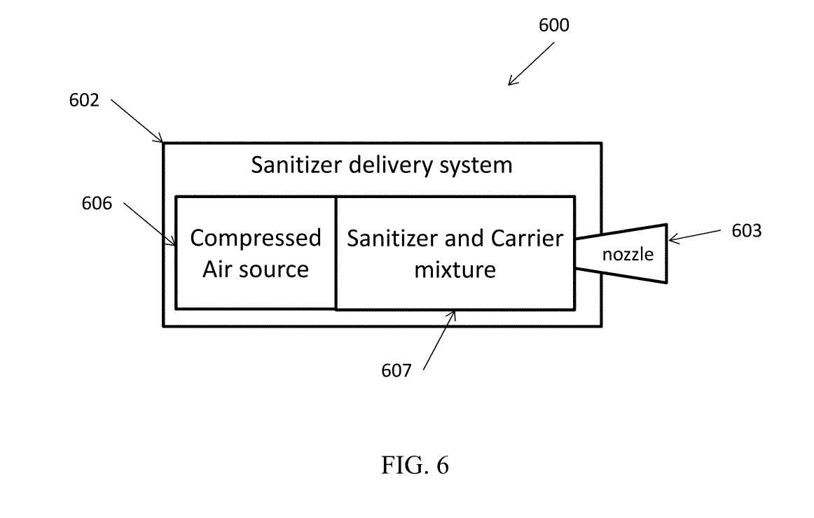

FIG. 6 is a block diagram conceptually illustrating an example system 600 for sanitizing an environment that includes multiple features, in accordance with certain aspects of the present disclosure. As shown a sanitizer delivery device 602 may be packaged such that all components may be provided in a single unit/system 600. Specifically, as shown, the sanitizer delivery device 602 may include a compressed air source 606 and a sanitizer and carrier mixture 607 internally. Further, the sanitizer delivery device 602 may include at least one nozzle 603 for releasing a mist.

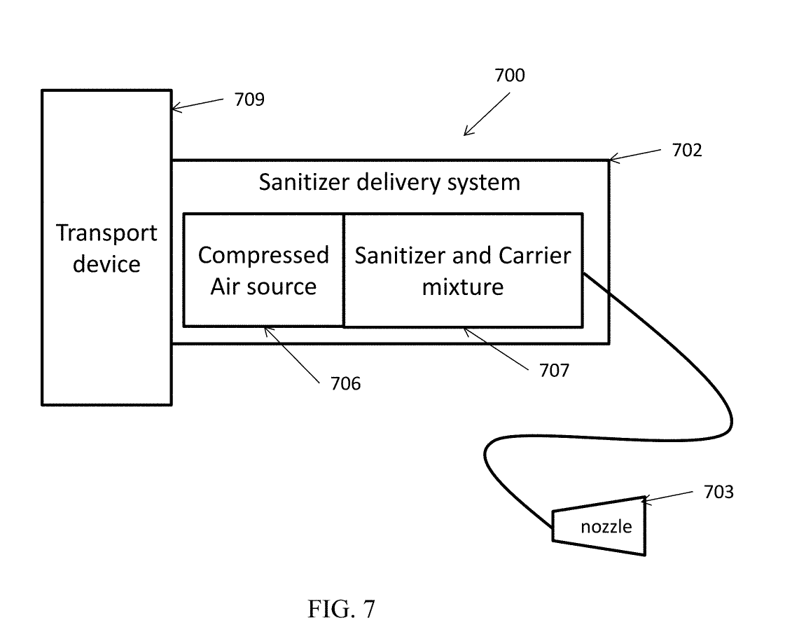

FIG. 7 is a block diagram conceptually illustrating an example system 700 for sanitizing an environment and/or target that includes multiple features, in accordance with certain aspects of the present disclosure. As shown a sanitizer delivery device 702 may be packaged such that all components may be provided in a single unit/system 700. Specifically, as shown, the sanitizer delivery device 702 may include a compressed air source 706 and a sanitizer and carrier mixture 707 internally. Further, the sanitizer delivery device 702 may include at least one nozzle 703 for releasing a mist. The nozzle 703 may be attached using a tube, hose, and/or cord. Additionally, the system 700 may include a transport device 709 which may be, for example, backpack straps which help provide a way to more easily move the system 700.

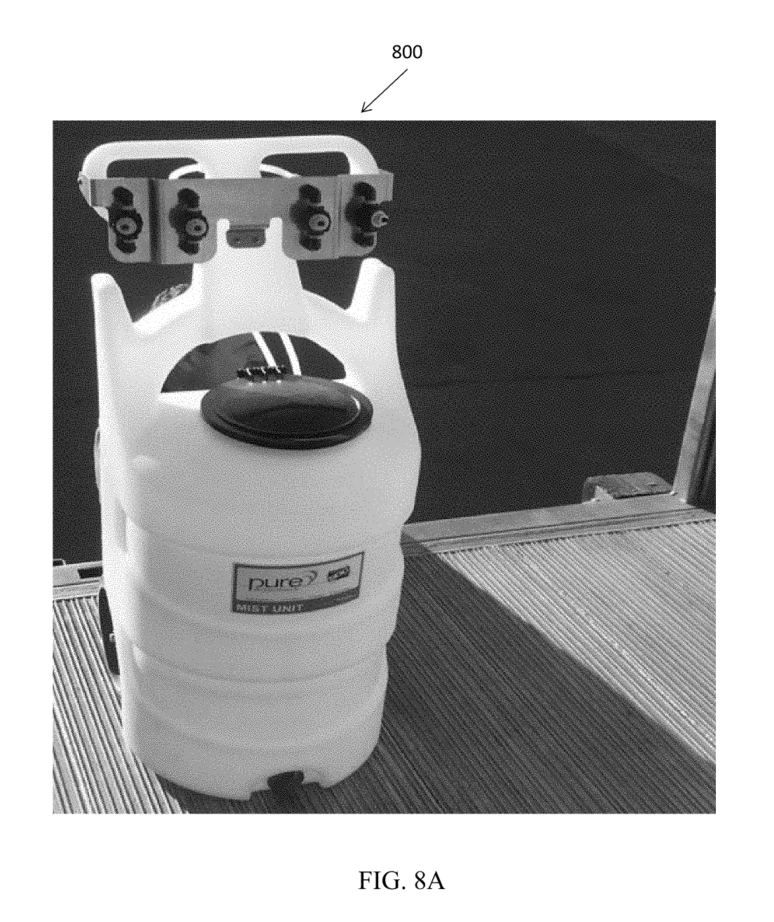

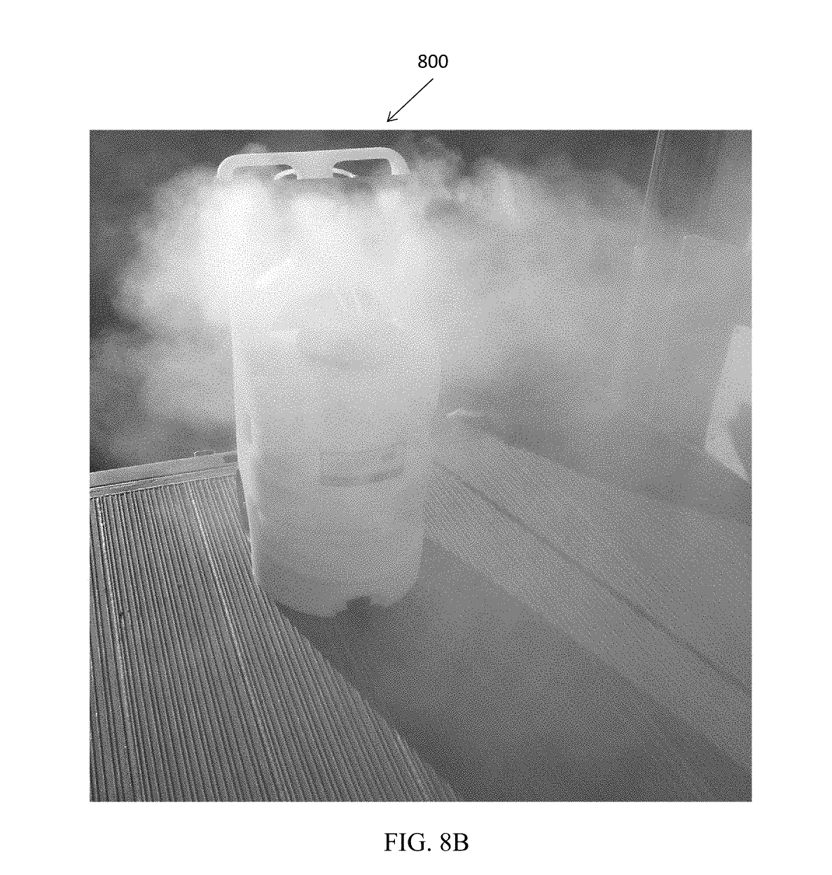

FIG. 8A illustrates a system 800 for sanitizing an environment and/or target that includes multiple features, in accordance with certain aspects of the present disclosure. As shown, the system 800 includes a tank and at last 4 nozzles for misting. FIG. 8B illustrates a system sanitizing an environment and/or target outputting a mist, in accordance with certain aspects of the present disclosure. A method of using the system 800 as shown in FIGS. 8A and 8B may include, but is not limited to: 1. Back truck up to dock; 2. Hook up the unit to Plant Air; 3. Place unit in the back of the truck; 4. Select truck size; and 5. Hit start and cycle will run for proper application.

### Example Methods of Sanitizing

In accordance with one or more cases, a method may be provided that provides a sanitizer and carrier mix for use with a sanitizer delivery device. The sanitizer delivery device may provide a mist of the sanitizer and carrier within a select area/environment such that the mist coats a target in the area. The target may include the walls, floor, and ceiling that define the environment space. The target may also include equipment or other objects that are found in the environment such as industrial food processing machines, pallets, tables, carts, barrels, and other objects and devices that may be found in any of the environments that are the target of sanitation.

For example, a 10-micron particle mist may be provided that includes silver ions as a sanitizer to control pathogens in a freeze tunnel environment where excess water would require an extensive interruption in processing. In another case, a similar fine mist may be used to sanitize cargo environments including, but not limited to, truck trailers, railcars, and shipping containers between uses without the need to rinse.

In one or more cases, a misting of sanitizer and carrier may be provided in an operations room that includes industrial equipment such as food processing equipment. The treatment time may be, for example, 17 minutes for the entire operations room and may consume a total of around two and a half gallons of sanitizer and carrier. The treatment time and consumption amount will vary depending on the size of the operations room and the type, number, and arrangement of any equipment in the operations room. Other factors that may be taken into account when misting in an operations room may include the ambient air properties as well as the current status of and operation of any air conditioning systems. Additionally, any opening in the environment may also be taken into the account when implementing a misting procedure.

Other cases where misting may be provided include other building spaces that may require sanitation such as hospital rooms including hospital equipment. Other environments where the disclosed sanitizing misting may be implemented may further include public transit vehicles including trains and buses, and public venues such as movie theaters, concert halls, kitchens, and other locations that may want to periodically sanitize.

Misting times may be based on the size of a room that is being sanitized. For example, in one case a 34,031 cubic foot room may be misted for 14 minutes. In another example, a 106,920 cubic foot room may be misted for a total of 12 minutes. These times may vary based on other factors such as the desired efficacy, the shape of the room, the equipment and items in the room, as well as other factors. For example, in the 34,031 cubic foot room, extra time may be allotted compared to the other room because the first room may have a mezzanine. In one or more cases, the misting may be done in spaces while the air conditioning (AC) systems remain on. In other cases, the AC system may be turned off initially while the area is initially filled with mist and then turned on while misting continues. This may provide a greater saturation prior to extensive air exchanges. Further, when misting in accordance with one or more examples described herein, some items in an environment may be covered such as any food or packaging.

In one or more other cases, a misting of sanitizer and carrier may be provided in a truck container that has a length of 20 feet. In another case, the truck container may be 40 feet. In these cases, the truck containers may be refrigerated containers used for transporting produce and other finished goods. The total misting time may vary depending on desired efficacy and time constraints. In one or more cases, the treatment time spent misting the truck container may be one minute for a 20-foot truck container and three minutes for the 40-foot truck container. The droplet size during misting may be, for example, 15 microns. Other micron scale droplet sizes may also be selected. This Ultra Fine Droplet Design (UFDD) provides a mist that does not cause an issue with adding moisture to the surfaces of targets or environments being sanitized. Further, in one or more cases, the mist may be sustained for a long period in a closed box allowing for continued microbial elimination and sanitation. In other cases, the environment may be a shipping container, a train cargo container, or any other known enclosed space that may desire sanitation.

Further, other length and dimensions of truck containers may also be selected as environments for sanitation. Also, the misting time may be adjusted even for similarly sized containers. For example, in other cases, a 53-foot container and a 40-foot container may be misted. Specifically, the 40-foot container may be treated for two minutes in this case while the larger 53-foot container may be treated for four minutes.

In one or more cases, the target for sanitation may be a handcart or a pallet that is used throughout an operation. For example, the cart or pallet may travel within an operations room, between one or more rooms, and onto and off of one or more truck containers. In this case, the handcart and the pallet may be misted using any one or more of the described misting devices. In accordance with one or more other cases, the target may be any other device or equipment such as a forklift.

In one or more cases specific targets, such as equipment, belts, scales, etc., may be selected as the target for sanitation. In these cases, the sanitizer delivery device may be a direct application device. The direct application device may be in the shape of a misting wand that includes nozzles at one end that disperse 15-micron droplet size mist. When misting specific equipment and devices, the misting time will vary depending on the size and complexity of the device. For example, the misting time may vary from 15 seconds to 3 minutes depending on the equipment/device. When spraying with the direct application device application may be done by moving at a constant rate along the equipment to help ensure homogenous coverage. Because there is no need to rinse or wash the equipment after, the sanitation process may be implemented during short breaks in usage such as during shift change, breaks, etc.

FIG. 9 illustrates operations 900 for sanitizing an environment, in accordance with aspects of the present disclosure. Specifically, operations 900 begin, at block 902, with providing a misting solution that includes a sanitizer. The operations 900 further include, at block 904, misting the misting solution into the environment such that a mist of the misting solution includes micron-sized particle mist for a predetermined misting time.

### Example Results and Testing

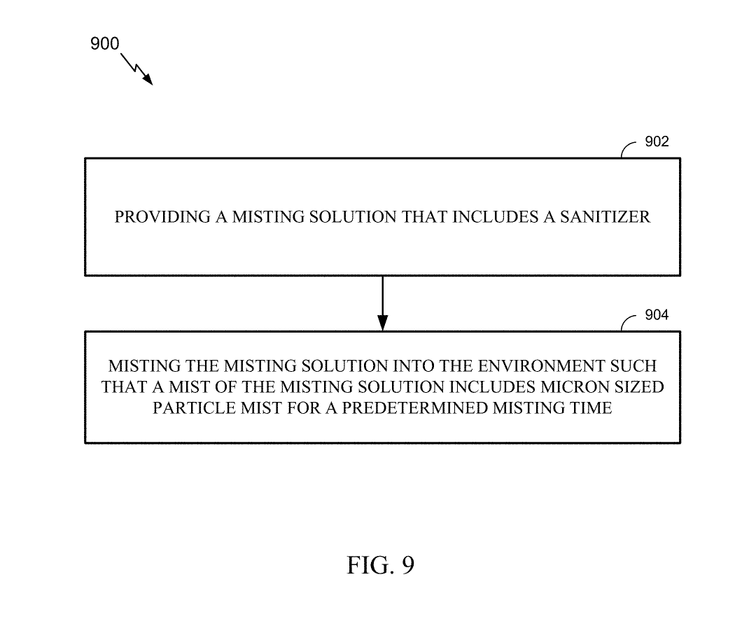

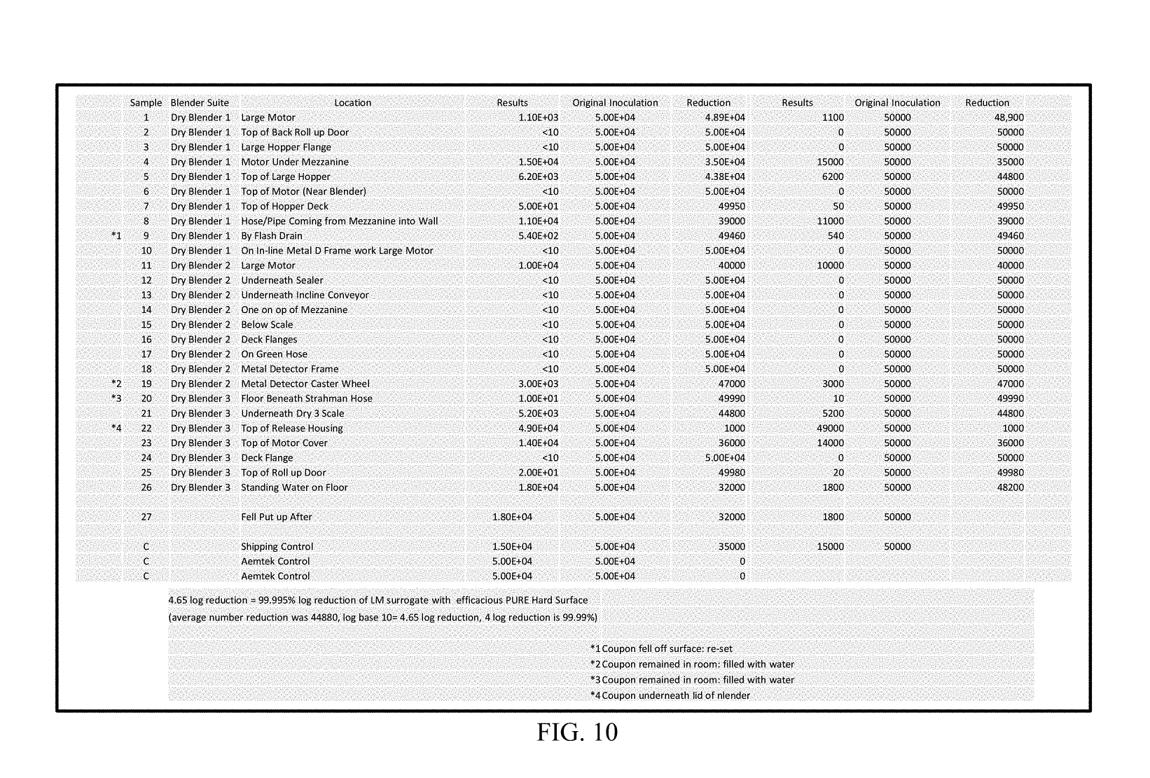

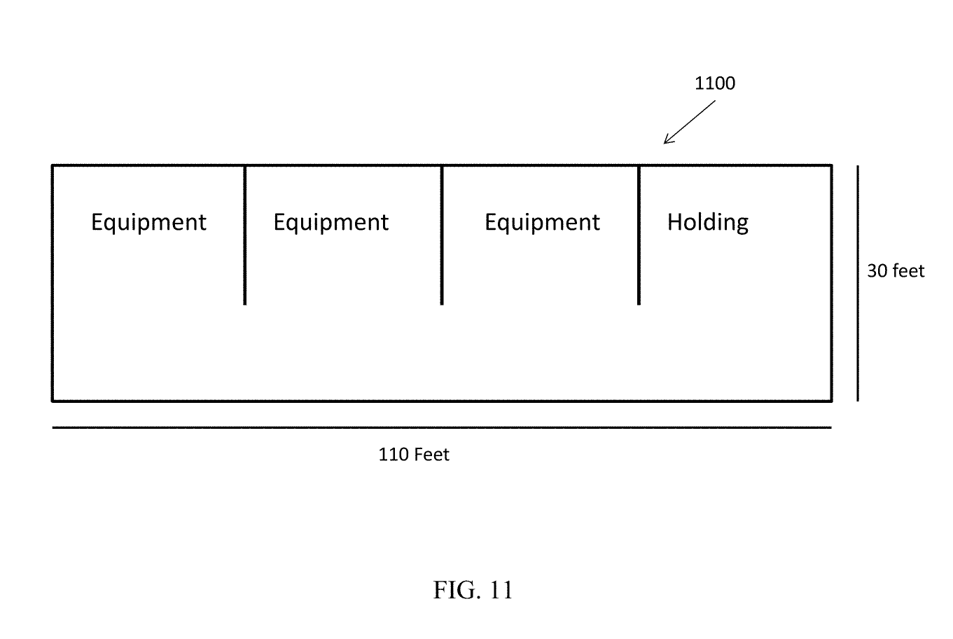

FIG. 10 is a table that contains sanitation results, in accordance with aspects of the present disclosure. FIG. 11 illustrates an environment within which the results in FIG. 10 were collected, in accordance with aspects of the present disclosure.

Specifically, the environment tested was a room with the following dimensions: Length 110 feet; Width 30 feet; and Height 20 feet with cubic feet 66,000 of space. The application time of the misting was around 2 minutes in each alcove for a total of about 8 minutes of misting using the misting unit. The amount of sanitizer and carrier mix that was used was about 2 gallons. The air that was supplied to the misting unit was plant air and was supplied at an available psi that was at least 55 psi.

The testing included a coupon based evaluation protocol. The protocol includes: Coupons inoculated with Listeria Monocytogenes surrogate: Pediococcus sp.; Stainless steel; 50,000 CFU per square inch test area; Shipping controls and lab retained control analysis performed; Coupons transported in Petri dishes placed in areas throughout processing alcoves; Petri dishes open just prior to misting and closed in each alcove following mist; Total exposure time 2 to 4 minutes; and Petri dishes closed and shipped overnight first delivery to lab.

In another testing example, an initial evaluation of a facility has been provided. Specifically, evaluation of various sites in the facility during pre-operation cleaning was implemented. Particularly, swabs were taken in an operating room following a normal cleaning cycle (control swabs) and following the sanitization using the misting sanitizer as discussed herein (test swabs). For this evaluation, the sanitizer was misted into the room using the misting unit. Total treatment time was 17 minutes for the entire operating room and about 2½ gallons of the sanitizer and carrier mix was used.

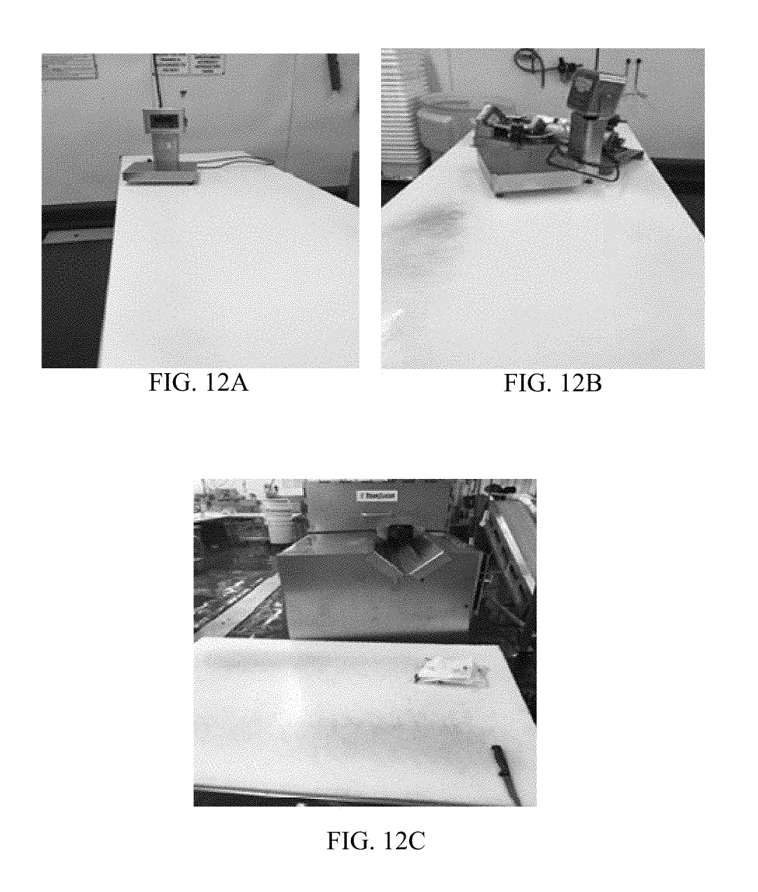

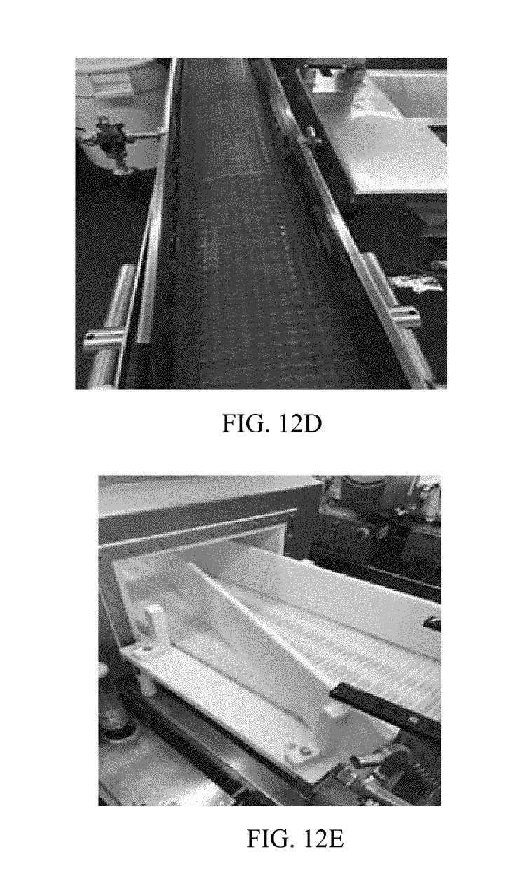

The location of swabs included, for example, working table surfaces and scale weighing surfaces (as shown in FIGS. 12A and 12B). Other locations were swabs were taken include, for example, other working table surfaces as shown in FIG. 12C, and a cleated product belt (shown in FIG. 12D), as well as a cleated product belt to metal detector area (shown in FIG. 12E).

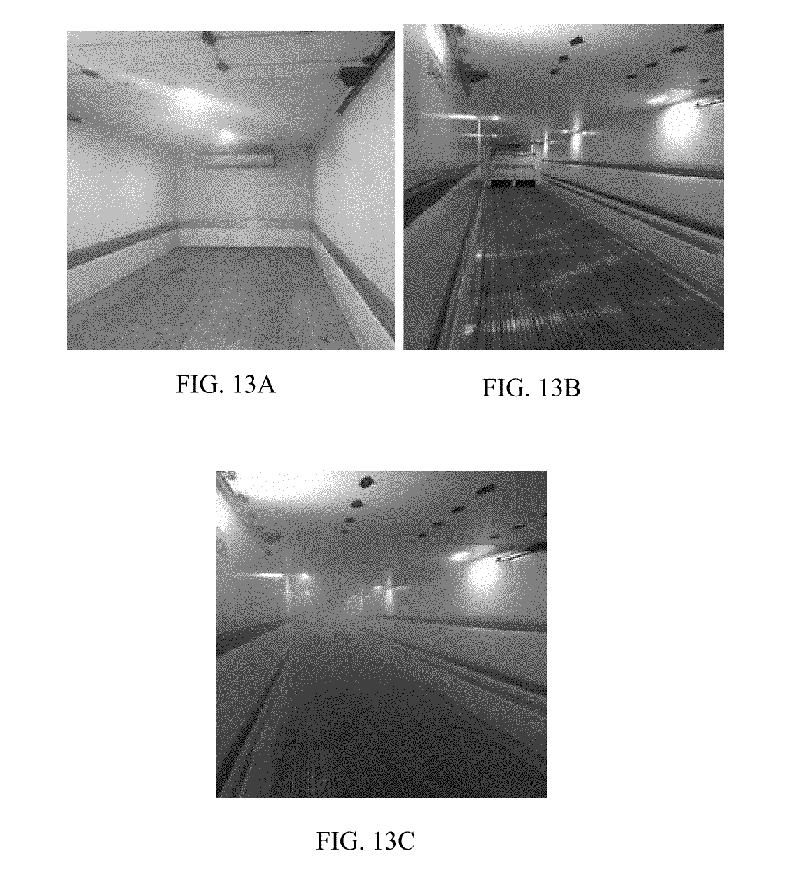

Other environments that may have been tested include trucks, particularly, the truck containers as shown FIGS. 13A, 13B, and 13C. Specifically, swabs were taken in two trucks that had been cleaned using the standard cleaning procedures and had not been sanitized; a 20-foot long box (FIG. 13A) and a 40-foot long box (FIG. 13B). Both trucks were refrigerated trucks used for produce and finished goods transport. All swabs were taken on the inside wall of the box. The control swabs were taken prior to the application of the misted sanitizer as disclosed herein and the test swabs taken 2 minutes following that application. The sanitizer and carrier mix was misted into the truck boxes using a misting unit. Total treatment time was 1 minute for the entire 20-foot box and 3 minutes for the 40-foot box.

Further, the sanitizer was also applied into the truck boxes using misting technology as described herein which provides a 15-micron-sized droplet. This Ultra Fine Droplet Design (UFDD) provides a mist that does not cause an issue with adding moisture to the surfaces. Further, this mist was sustained for a long period in a closed box allowing for continued kill as shown in FIG. 13C which is an example 15 minutes after mist application.

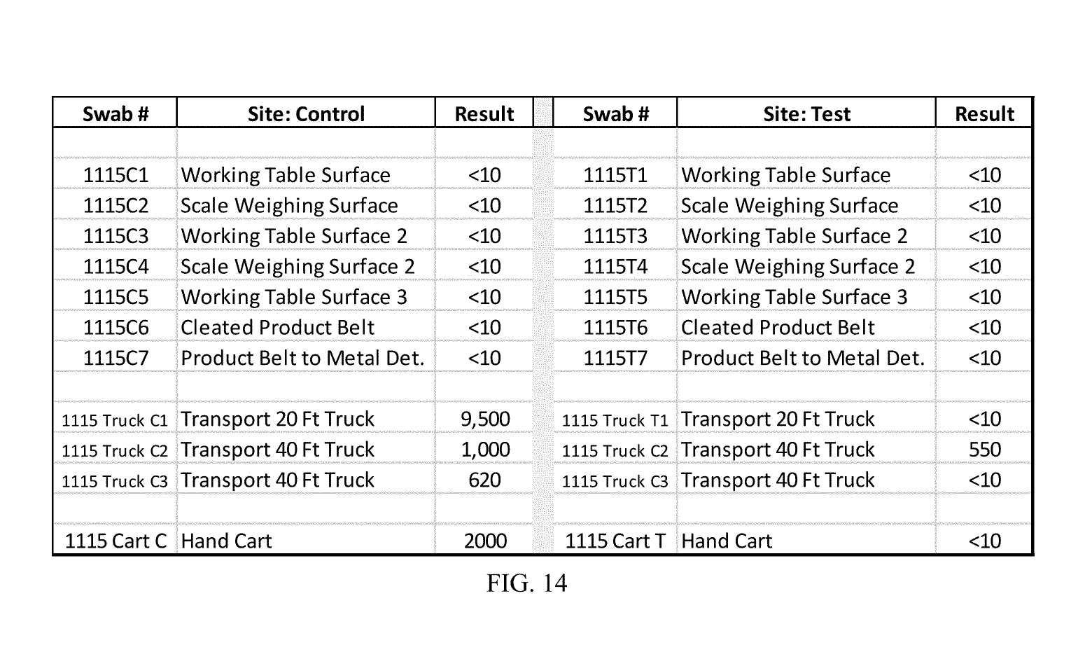

Another place that was tested includes handcarts. Multiple hand carts are used throughout an operation. These carts were evaluated for the effectiveness of the sanitizer. For example, carts that were cleaned according to standard cleaning procedures were swabbed as a control and then the misted sanitizer was applied and swabs taken following that application. These swabs were taken from the handle area on top of the cart. Results from all of these swab location and testing procedures is shown in the table shown in FIG. 14. Specifically, swab 1115 Cart C is shown with results of 2000 colony-forming units (CFU) when swabbed as a control after standard cleaning procedures. Then, after application of the sanitizer as described herein, a swab 1115 Cart T of that hand card shows that the handcart now only has less than 10 colony-forming units (CFU).

FIG. 14 also shows testing results of other targets and environments. For example, a transport truck with a 20 ft space showed 9,500 CFU when swabbed as a control and then after sanitizer was applied the transport truck showed less than 10 CFU.

Another facility underwent two separate evaluations at two different times using two different application methodologies. In both cases, the evaluation was completed using inoculated pitted stainless-steel coupons. These coupons were inoculated at 5.4+05 Pediococcus spp. (NRRL B-2354: Enterococcus faecium) as a surrogate for Listeria monocytogenes.

The coupons inoculated, affixed then sent overnight in a cooler to the test location. The coupons are affixed to a sterile petri dish and covered. Upon receipt, at the test location, the coupons were placed in the locations through the facility and marked with the location. Just prior to application of the misted sanitizer as described herein the lids of the Petri dishes were removed. Following treatment (approximately 2 to 5 minutes) after application the dishes were again closed, gathered, places back into a cooler and sent overnight back to laboratories for testing.

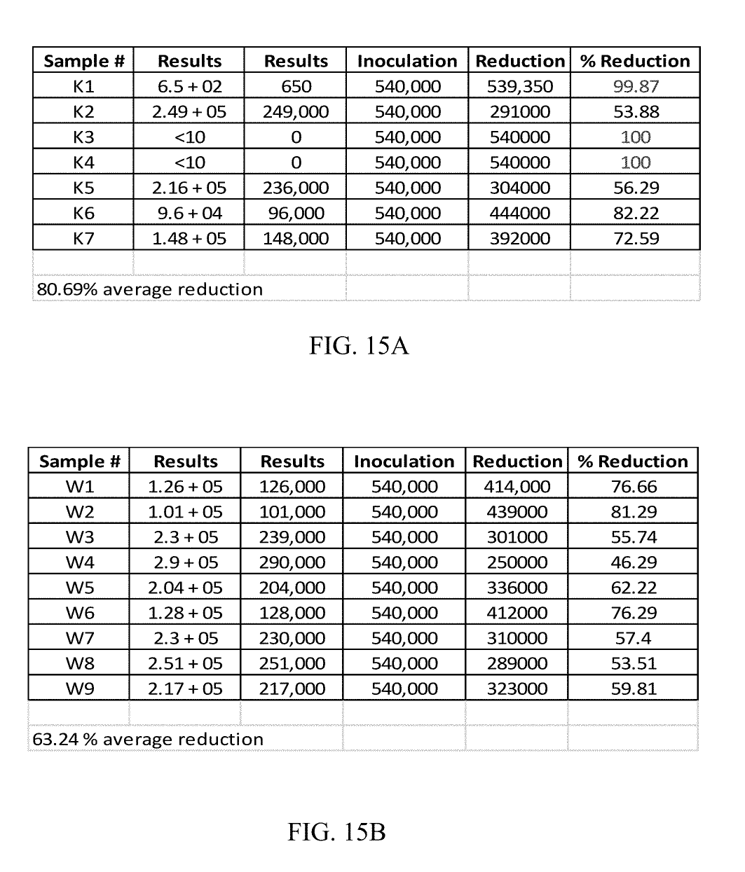

In a first evaluation, there were two areas within the location tested; MOD 2 and Kitchen 1 (K1). The application of the sanitizer was done by misting the entire room using a misting unit equipped with nozzles that produce a 15-micron-sized droplet mist. K1 total misting time was 14 minutes for a 34,031 cubic feet room. For MOD 2, a total misting time was 12 minutes for a 106,920 cubic feet room. Air conditioning in the two areas was kept on. Extra time was allowed in the K1 area due to the location and presence of a mezzanine. No personal protective equipment (PPE) may be required but food and packaging must be covered. The results of this first evaluation testing are shown in FIG. 15A for the kitchen 1 and 15B for the MOD 2 space. For example, for a sample K1 in the kitchen 1 an initial inoculation of 540,000 colony-forming units (CFU) was present. Then after application of the sanitizer K1 results showed 650 CFU providing a reduction of 539,350 CFU which is a 99.87 reduction. In the MOD 2 space, a sample W1 showed a reduction of 414,000 CFU from 540,000 CFU for a 76.66% reduction.

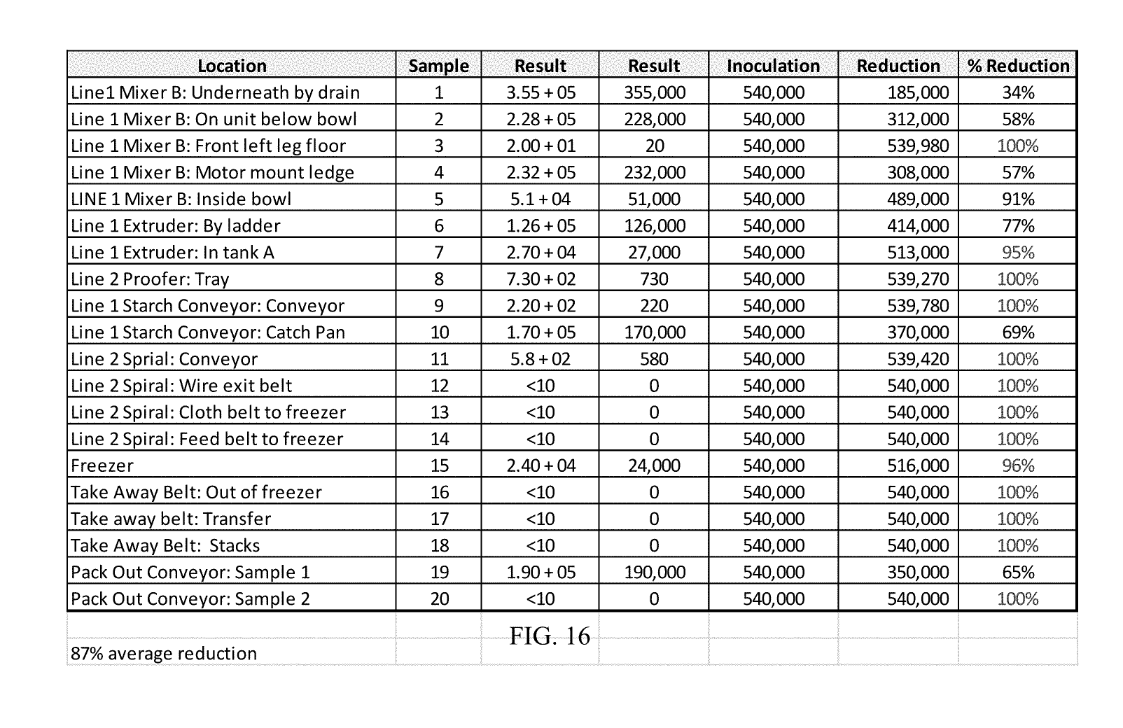

In the second evaluation, the sanitizer application was accomplished using a “direct apply” misting wand. This wand is equipped with the same 15-micron droplet size producing nozzle as the previous evaluation but is designed to be directed at equipment, belts, scales etc. rather than mist an entire room. The evaluation was completed on an entire product line from mixing—extrusion—proofing—starching spiral cooler—freezer—packaging belts. The misting was completed in each area as it would on a standard SOP. Misting time varied by the size of the equipment treated; ranging from 15 seconds to 3 minutes. When using the direct apply the person applying moves at a constant rate along the equipment ensuring a homogeneous coverage. No PPE is required but food and packaging must be covered. The results of this second evaluation are shown in FIG. 16.

As shown, both application methodologies provide an efficacy against the LM Surrogate. The data suggests that in the first evaluation in the Mod 2 area the time allowed for treatment may not have been optimized due to the air movement in the area at the time of the test. In future SOPs the sequence may include filling the room with mist (possibly 5 minutes) first, then turning the air on and continuing the misting for the remaining time, this may allow for a greater saturation prior to extensive air exchanges. Further, as shown in FIG. 16, the “direct apply” wand provided the greatest efficacy with an 87% overall averaged reduction with over ½ of the coupons showing complete elimination of the surrogate organism.

A combination of the two application methodologies may also be provided. For example, every cleaning cycle or desired kill step the directed wand is used to mist the lines; this can be done during shift change, breaks etc. as the sanitizer is non-toxic and does not need to be rinsed from the line (even food contact surfaces) only food and packaging need to be covered. Then once per week (or up to 10 days in some partner cases) the area is completely misted using the area misting head. This allows a complete saturation of the area, equipment, walls, floors etc.

In one or more cases, a system for providing microbial sanitation is provided. The system comprises a sanitizer configured to sanitize at least one of an environment and a target. The system also comprises a carrier configured to mix with the sanitizer creating a liquid mixture of sanitizer and carrier. Further, the system comprises a sanitizer delivery device configured to hold the liquid mixture, receive and/or apply pressurized air to the liquid mixture, and release the pressurized liquid mixture through a nozzle that creates a mist with micron-sized liquid particles that contact one or more of the environment and the target for a predetermined misting time based on at least one of the environment or the target.

In some cases, the sanitizer includes one or more of silver ions and plasma activated water. In some cases, the environment is one or more of a freezer tunnel, a trailer, a truck container, a cargo ship container, an operations room, railcars, and an enclosed space. In some cases, the target is one or more of industrial equipment, a medical device, industrial food processing machines, a pallet, a table, a cart, a barrel, and other objects and devices that are found in the environment. In some cases, the carrier is water. In some cases, the sanitizer delivery device is further configured to concentrate the sanitizer onto surfaces to be sanitized. In some cases, the sanitizer is an environmentally compatible sanitizer.

In some cases, the predetermined misting time is determined based on one or more factors, wherein the factors include one or more of environment size, environment shape, air quality, air flow, environment openings, target properties, target location, number of targets, system location, system placement, system arrangement, and mist flow. In some cases, the predetermined misting time ranges from 15 seconds to 20 minutes.

In some cases, the system further comprises a lift device configured to lift the sanitizer delivery device to a select distance above a lower surface of the environment the system is in. In some cases, the system further comprises a transport device configured to assist the system is moving from one position to another. The transport device includes one or more of push wheels, backpack straps, motorized tracks, and motorized wheels. In some cases, the system further comprises a plurality of nozzles configured to simultaneously release mist.

In one or more cases, a sanitizer delivery device is provided. The sanitizer delivery device comprises a sanitizer and carrier mixture reservoir configured to hold a sanitizer and carrier mixture in liquid form that is configured to be misted. The sanitizer delivery device also comprises a compressed air source that is configured to apply compressed air to the sanitizer and carrier mixture. Further, the sanitizer delivery device comprises at least one nozzle that is configured to create a mist from the sanitizer and carrier mixture as the sanitizer and carrier mixture passes through the nozzle under pressure from the compressed air source that is applied.

In some cases, a method for sanitizing an environment is provided. The method comprises providing a misting solution that includes a sanitizer and misting the misting solution into an environment such that a mist of the misting solution includes micron-sized particle mist for a predetermined misting time based on at least one of the environment or a target in the environment.

In some cases, the environment is a low moisture environment. In some cases, the misting solution further comprises a carrier. In some cases, the sanitizer comprises silver ions. In some cases, the sanitizer comprises plasma activated water. In some cases, the micron-sized particle mist includes particles ranging from 5 to 25 microns in diameter. In some cases, the micron-sized particle mist includes particles that are 10 microns in diameter. In some cases, the micron-sized particle mist includes particles that are 15 microns in diameter.

In some cases, the environment is one or more of a freezer tunnel, a trailer, a truck container, a cargo ship container, an operations room, railcar, and an enclosed space. In some cases, the target is one or more of industrial equipment, a medical device, industrial food processing machines, a pallet, a table, a cart, a barrel, and other objects and devices that are found in the environment.

In some cases, the method further comprises concentrating the sanitizer onto surfaces to be sanitized. In some cases, the sanitizer is an environmentally compatible sanitizer. In some cases, the predetermined misting time is determined based on one or more factors, wherein the factors include one or more of environment size, environment shape, air quality, air flow, environment openings, target properties, target location, number of targets, system location, system placement, system arrangement, and mist flow. In some cases, the predetermined misting time ranges from 15 seconds to 20 minutes.

In some cases, the method further comprises lifting, using a lift device, a sanitizer delivery device configured to mist the misting solution to a select distance above a lower surface of the environment the sanitizer delivery device is in. In some cases, the method further comprises moving from one position to another using a transport device. In some cases, the transport device includes one or more of push wheels, backpack straps, motorized tracks, and motorized wheels. In some cases, the method further comprises simultaneously releasing mist using a plurality of nozzles for the predetermined misting time.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). As used herein, including in the claims, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” For example, the articles “a” and “an” as used in this application and the appended claims should generally be construed to mean “one or more” unless specified otherwise or clear from the context to be directed to a singular form. Unless specifically stated otherwise, the term “some” refers to one or more. Moreover, the term “or” is intended to mean an inclusive “or” rather than an exclusive “or.” That is, unless specified otherwise or clear from the context, the phrase, for example, “X employs A or B” is intended to mean any of the natural inclusive permutations. That is, for example the phrase “X employs A or B” is satisfied by any of the following instances: X employs A; X employs B; or X employs both A and B. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

It is to be understood that the claims are not limited to the precise configuration and components illustrated above. Various modifications, changes and variations may be made in the arrangement, operation and details of the methods and apparatus described above without departing from the scope of the claims.

What is claimed is:

1. A system for providing microbial sanitation, the system comprising:
a sanitizer configured to sanitize at least one of an environment or a target;
a carrier configured to mix with the sanitizer creating a liquid mixture of sanitizer and carrier; and
a sanitizer delivery device configured to:
hold the liquid mixture,
apply pressurized air to the liquid mixture, and
release the pressurized liquid mixture through a nozzle that creates a mist with micron-sized liquid particles that contact one or more of the environment or the target for a predetermined misting time based on at least one of the environment or the target.

2. The system of claim 1, wherein the sanitizer includes one or more of silver ions or plasma activated water.

3. The system of claim 1, wherein the environment is one or more of a freezer tunnel, a trailer, a truck container, a cargo ship container, an operations room, railcars, or an enclosed space.

4. The system of claim 1, wherein the target is one or more of industrial equipment, a medical device, industrial food processing machines, a pallet, a table, a cart, a barrel, or other objects and devices that are found in the environment.

5. The system of claim 1, wherein the carrier is water.

6. The system of claim 1, wherein the sanitizer delivery device is further configured to concentrate the sanitizer onto surfaces to be sanitized.

7. The system of claim 1, wherein the sanitizer is an environmentally compatible sanitizer.

8. The system of claim 1, wherein the predetermined misting time is determined based on one or more factors, wherein the factors include one or more of environment size, environment shape, air quality, air flow, environment openings, target properties, target location, number of targets, system location, system placement, system arrangement, or mist flow.

9. The system of claim 8, wherein the predetermined misting time ranges from 15 seconds to 20 minutes.

10. The system of claim 1, further comprising:
a lift device configured to lift the sanitizer delivery device to a select distance above a lower surface of the environment the system is in.

11. The system of claim 1, further comprising:
a transport device configured to assist the system in moving from one position to another,
wherein the transport device includes one or more of push wheels, backpack straps, motorized tracks, or motorized wheels.

12. The system of claim 1, further comprising:
a plurality of nozzles configured to simultaneously release mist.

13. A sanitizer delivery device that comprises:
a sanitizer and carrier mixture reservoir configured to hold a sanitizer and carrier mixture in liquid form that is configured to be misted;
a compressed air source that is configured to apply compressed air to the sanitizer and carrier mixture; and
at least one nozzle that is configured to create a mist from the sanitizer and carrier mixture as the sanitizer and carrier mixture passes through the nozzle under pressure from the compressed air source that is applied.

14. A method for sanitizing an environment comprising:
providing a misting solution that includes a sanitizer; and
misting the misting solution into an environment such that a mist of the misting solution includes micron-sized particle mist for a predetermined misting time based on at least one of the environment or a target in the environment.

15. The method of claim 14, wherein the environment is a low moisture environment.

16. The method of claim 14, wherein the misting solution further comprises a carrier.

17. The method of claim 14, wherein the sanitizer comprises silver ions.

18. The method of claim 14, wherein the sanitizer comprises plasma activated water.

19. The method of claim 14, wherein the micron-sized particle mist includes particles ranging from 5 to 25 microns in diameter.

20. The method of claim 14, wherein the micron-sized particle mist includes particles that are 10 microns in diameter.

21. The method of claim 14, wherein the micron-sized particle mist includes particles that are 15 microns in diameter.

22. The method of claim 14, wherein the environment is one or more of a freezer tunnel, a trailer, a truck container, a cargo ship container, an operations room, railcar, or an enclosed space.

23. The method of claim 14, wherein the target is one or more of industrial equipment, a medical device, industrial food processing machines, a pallet, a table, a cart, a barrel, or other objects and devices that are found in the environment.

24. The method of claim 14, further comprising concentrating the sanitizer onto surfaces to be sanitized.

25. The method of claim 14, wherein the sanitizer is an environmentally compatible sanitizer.

26. The method of claim 14, wherein the predetermined misting time is determined based on one or more factors, wherein the factors include one or more of environment size, environment shape, air quality, air flow, environment openings, target properties, target location, number of targets, system location, system placement, system arrangement, or mist flow.

27. The method of claim 26, wherein the predetermined misting time ranges from 15 seconds to 20 minutes.

28. The method of claim 14, further comprising:
lifting, using a lift device, a sanitizer delivery device configured to mist the misting solution to a select distance above a lower surface of the environment the sanitizer delivery device is in.

29. The method of claim 14, further comprising:
moving from one position to another using a transport device,
wherein the transport device includes one or more of push wheels, backpack straps, motorized tracks, or motorized wheels.

30. The method of claim 14, further comprising:
simultaneously releasing mist using a plurality of nozzles for the predetermined misting time.
